(19) United States
(12) Patent Application Publication
(10) Pub. No.: US 2026/0041656 A1
(43) Pub. Date: Feb. 12, 2026

(54) TREATING PULMONARY HYPERTENSION BASED ON NOVEL BIOMARKER

(71) Applicant: United Therapeutics Corporation, Silver Spring, MD (US)

(72) Inventors: Kenta Takahashi, Tokyo (JP); Atsuko Murakami, Tokyo (JP)

(73) Assignee: United Therapeutics Corporation, Silver Spring, MD (US)

(21) Appl. No.: 19/290,984

(22) Filed: Aug. 5, 2025

Related U.S. Application Data
(60) Provisional application No. 63/679,951, filed on Aug. 6, 2024.

(51) Int. Cl. A61K 31/192; A61K 9/00; A61P 11/00; G01N 33/68

(52) U.S. Cl. CPC A61K 31/192; A61K 9/0073; A61P 11/00; G01N 33/6884; G01N 2333/46; G01N 2800/52

(57) ABSTRACT

Provided are novel methods of treating a subject having increased expression of a KL-6 gene and suffering from lung disease comprising administering an effective amount of a vasodilator to the subject

### CROSS-REFERENCE TO RELATED APPLICATIONS

The present application claims priority to U.S. Provisional Application No. 63/679,951, filed Aug. 6, 2024, the entire contents of which are incorporated herein by reference.

### TECHNICAL FIELD

The present application relates to methods for therapeutic treatment and, more particularly, to therapeutic methods involving increased KL-6 gene expression and lung disease.

### BACKGROUND

Pulmonary hypertension (PH) due to lung disease or hypoxia is classified as ‘Group 3’ in the World Health Organization classification of PH.¹ In idiopathic pulmonary fibrosis, a mean pulmonary arterial pressure (mPAP)≥25 mmHg has been reported in 8-15% of patients upon initial work-up, with greater prevalence in advanced (30-50%) and end-stage (>60%) disease.¹ PH-ILD results in reduced exercise capacity, greater supplemental oxygen requirements, decreased quality of life, and a worse prognosis compared to ILD alone.² Therefore, early diagnosis and appropriate treatment by specialists are important. Although data are limited, therapies approved for PAH (Group 1) have been used to treat Group 3. In a registry study of Group 3 in Japan, it was reported that 56-76% of patients had received PAH-specific therapy.³ However, several clinical trials failed to convincingly demonstrate a clinical benefit of pulmonary vasodilators in various PH-ILD populations^{4,5}. Further, there is limited data regarding the specific biomarkers indicating the progression and treatment for lung disease, and PH-ILD in particular.

Accordingly, there is currently a need for identifying patients that would benefit from pulmonary vasodilation therapy.

### SUMMARY

Applicant has discovered the KL-6 expression is correlated with in some, but not all cases of lung disease and/or dysfunction. Kerbs von Lungren 6 antigen (KL-6) is a high molecular weight glycolprotein encoded by the MUC1 gene and is found on the cell surface of type II alveolar cells. Disclosed herein are methods of improving lung function and decreasing KL-6 expression in patients suffering from lung disease or dysfunction, and in particular PH-ILD. The methods disclosed herein include the administration of an effective amount of a vasodilator, and in particular Treprostinil, to an individual overexpressing KL-6.

In one aspect, a method of treating a subject having increased expression of KL-6 and suffering from lung disease or lung dysfunction is disclosed. In yet another aspect, a method of treating lung disease or lung dysfunction in a subject suffering therefrom and having an increased expression of KL-6 is disclosed. In yet another aspect, a method of reducing KL-6 expression in a subject suffering from lung disease or lung dysfunction. In yet another aspect, a method of treating lung disease or lung dysfunction in a subject suffering therefrom and having previously been determined as having increased expression of KL-6 is disclosed.

In some aspects, the methods comprise, consist of, or consist essentially of administering an effective amount of a vasodilator to the subject. In some aspects, the vasodilator comprises, consists of, or consists essentially of Treprostinil or a salt thereof. In some aspects, the effective amount of the vasodilator is an amount sufficient to reduce KL-6 expression. In some aspects, the effective amount of the vasodilator is an amount sufficient to decease KL-6 expression to below 600 U/ml, or below 650 U/ml, or below 700 U/ml or below 750 U/ml. In some aspects, the Treprostinil or salt thereof is administered by inhalation at a concentration of about 500 μg/ml to about 2500 μg/ml. In some aspects, the effective amount of the vasodilator is an amount sufficient to reduce KL-6 expression and increase forced vital capacity above 2.05 L, or 2.1 L, or 2.15 L, or 2.2 L. In some aspects, the effective amount is between 5-500 μg inhaled Treprostinil per day. In some aspects, administration of the effective amount of the vasodilator results in increased force vital capacity in the subject.

In some aspects, the method further comprise, consist of, or consist essentially of the step of detecting the level of KL-6 expression prior to administration of the vasodilator. In some aspects, the method further comprise, consist of, or consist essentially of the step of detecting the level of KL-6 expression following administration of the vasodilator.

In some aspects, the lung disease comprises, consists of, or consists essentially of pulmonary hypertension associated with interstitial lung disease (PH-ILD).

In some aspects, the patient exhibits a baseline expression of KL-6 that is greater than 600 U/ml, or greater than 650 U/ml, or greater than 700 U/ml, or greater than 750 U/ml, or greater than 800 U/ml prior to administration of the vasodilator. In some aspects, the subject exhibits a baseline forced vital capacity of less than 2.20 L, or less than 2.15 L, or less than 2.10 L prior to administration of the vasodilator.

In some aspects, the vasodilator is administered by intravenous administration, intramuscular administration, oral administration, or inhalation.

In some aspects, the subject is a mammalian subject. In yet another aspect, the subject is a human subject. In some aspects, the subject has mean pulmonary arterial pressure (mPAP)≥25 mmHg and a pulmonary arterial wedge pressure (PAWP)≤15 mmHg as confirmed by a baseline right heart catheterization (RHC). In yet another aspect, the subject is suffering from interstitial pneumonia.

### BRIEF DESCRIPTION OF THE DRAWINGS

FIG. 1: Change in peak 6MWD from baseline to week 52. Data shown as the median and 95% CI. The data shown are for patients with available data (observed), but also include missing data imputed analysis for the 16-week time point. In the primary analysis of the peak 6MWD, the 6MWD was set to 0 m for subjects who had no 6MWD measurement at week 16 due to death or worsening of ILD or PH. Last Observation Carried Forward (LOCF) or Baseline Observation Carried Forward (BOCF) was used for imputation for all other subjects who did not have the 6MWD measurement at week 16. Abbreviations: BOCF, Baseline Observation Carried Forward; CI, confidence interval; ILD, interstitial lung disease; LOCF, Last Observation Carried Forward; PH, pulmonary hypertension; 6MWD, 6-minute walking distance.

FIG. 2: Plasma treprostinil concentration versus time following administration of 72 μg of inhaled treprostinil (n=15). Data shown as the mean±SD.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. Although not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety. In case of conflict, the present specification, including definitions, will control. Other aspects are set forth within the claims that follow.

Unless the context indicates otherwise, it is specifically intended that the various features described herein can be used in any combination. Moreover, the disclosure also contemplates that in some embodiments, any feature or combination of features set forth herein can be excluded or omitted. To illustrate, if the specification states that a complex comprises components A, B, and C (or A, B, and/or C), it is specifically intended that any of A, B or C, or a combination thereof, can be omitted and disclaimed singularly or in any combination.

All numerical designations, e.g., pH, temperature, time, concentration, and molecular weight, including ranges, are approximations that can be varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”.

### Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, “optional” or “optionally” means that the subsequently described event or circumstance can or cannot occur, and that the description includes instances where the event or circumstance occurs and instances where it does not.

Interstitial lung disease (ILD) describes a large group of disorders, most of which cause progressive scarring of lung tissue. The scarring associated with interstitial lung disease eventually affects your ability to breathe and get enough oxygen into your bloodstream. Interstitial lung disease can be caused by long-term exposure to hazardous materials, such as asbestos. Some types of autoimmune diseases, such as rheumatoid arthritis, also can cause interstitial lung disease. In some cases, however, the causes remain unknown.

Patient's suffering from ILD often suffer from or develop pulmonary hypertension.

Pulmonary hypertension may occur due to various reasons and the different entities of pulmonary hypertension were classified based on clinical and pathological grounds in 5 categories according to the latest WHO convention, see e.g. Simonneau G., et al. J. Am. Coll. Cardiol. 2004; 43(12 Suppl S):5S-12S. Pulmonary hypertension can be a manifestation of an obvious or explicable increase in resistance, such as obstruction to blood flow by pulmonary emboli, malfunction of the heart's valves or muscle in handling blood after its passage through the lungs, diminution in pulmonary vessel caliber as a reflex response to alveolar hypoxia due to lung diseases or high altitude, or a mismatch of vascular capacity and essential blood flow, such as shunting of blood in congenital abnormalities or surgical removal of lung tissue. In addition, certain infectious diseases, such as HIV and liver diseases with portal hypertension may cause pulmonary hypertension. Autoimmune disorders, such as collagen vascular diseases, also often lead to pulmonary vascular narrowing and contribute to a significant number of pulmonary hypertension patients. The cases of pulmonary hypertension remain where the cause of the increased resistance is as yet inexplicable are defined as idiopathic (primary) pulmonary hypertension (iPAH) and are diagnosed by and after exclusion of the causes of secondary pulmonary hypertension and are in the majority of cases related to a genetic mutation in the bone morphogenetic protein receptor-2 gene. The cases of idiopathic pulmonary arterial hypertension tend to comprise a recognizable entity of about 40% of patients cared for in large specialized pulmonary hypertension centers. Approximately 65% of the most commonly afflicted are female and young adults, though it has occurred in children and patients over 50. Life expectancy from the time of diagnosis is short without specific treatment, about 3 to 5 years, though occasional reports of spontaneous remission and longer survival are to be expected given the nature of the diagnostic process. Generally, however, disease progress is inexorable via syncope and right heart failure and death is quite often sudden.

Pulmonary hypertension refers to a condition associated with an elevation of pulmonary arterial pressure (PAP) over normal levels. In humans, a typical mean PAP is approximately 12-15 mm Hg. Pulmonary hypertension, on the other hand, can be defined as mean PAP above 25 mmHg, assessed by right heart catheter measurement. Pulmonary arterial pressure may reach systemic pressure levels or even exceed these in severe forms of pulmonary hypertension. When the PAP markedly increases due to pulmonary venous congestion, i.e. in left heart failure or valve dysfunction, plasma can escape from the capillaries into the lung interstitium and alveoli. Fluid buildup in the lung (pulmonary edema) can result, with an associated decrease in lung function that can in some cases be fatal. Pulmonary edema, however, is not a feature of even severe pulmonary hypertension due to pulmonary vascular changes in all other entities of this disease.

Pulmonary hypertension may either be acute or chronic. Acute pulmonary hypertension is often a potentially reversible phenomenon generally attributable to constriction of the smooth muscle of the pulmonary blood vessels, which may be triggered by such conditions as hypoxia (as in high-altitude sickness), acidosis, inflammation, or pulmonary embolism. Chronic pulmonary hypertension is characterized by major structural changes in the pulmonary vasculature, which result in a decreased cross-sectional area of the pulmonary blood vessels. This may be caused by, for example, chronic hypoxia, thromboembolism, collagen vascular diseases, pulmonary hypercirculation due to left-to-right shunt, HIV infection, portal hypertension or a combination of genetic mutation and unknown causes as in idiopathic pulmonary arterial hypertension.

Pulmonary hypertension has been implicated in several life-threatening clinical conditions, such as adult respiratory distress syndrome (“ARDS”) and persistent pulmonary hypertension of the newborn (“PPHN”). Zapol et al., Acute Respiratory Failure, p. 241-273, Marcel Dekker, New York (1985); Peckham, J. Ped. 93:1005 (1978). PPHN, a disorder that primarily affects full-term infants, is characterized by elevated pulmonary vascular resistance, pulmonary arterial hypertension, and right-to-left shunting of blood through the patent ductus arteriosus and foramen ovale of the newborn's heart. Mortality rates range from 12-50%. Fox, Pediatrics 59:205 (1977); Dworetz, Pediatrics 84:1 (1989). Pulmonary hypertension may also ultimately result in a potentially fatal heart condition known as “cor pulmonale,” or pulmonary heart disease. Fishman, “Pulmonary Diseases and Disorders” 2^{nd} Ed., McGraw-Hill, New York (1988).

Kerbs von Lungren 6 antigen (KL-6) is a high molecular weight glycolprotein encoded by the MUC1 gene and is found on the cell surface of type II alveolar cells. Applicant's discovered that elevated levels of KL-6 proteins in subjects correlated with decreased lung function and the presence of lung diseases, such as PH-ILD. However, it is important to note that KL-6 may not be elevated in every subject suffering from lung diseases or disorders. However, Applicant's discovered that modulation and reduction of KL-6 expression can lead to increase lung function in some patients suffering from lung disease. In some aspects, baseline levels of KL-6 in the subjects suffering from lung disease or lung disorders may be greater than 600 U/ml, or greater than 650 U/ml, or greater than 700 U/ml, or greater than 750 U/ml, or greater than 800 U/ml. In some aspects, administration of an effective amount of a vasodilator results in the decrease of KL-6 expression below baseline levels. In some aspects, KL-6 expression is decreased below 600 U/ml, or below 650 U/ml, or below 700 U/ml or below 750 U/ml.

Vasodilators as provided herein are a class of therapeutic drugs capable of dilating the blood vessels, thus enabling increased blood flow in a particular area of the body. In some aspects, the vasodilators provided herein are vasodilators that increase blood flow and vasodilation of the blood vessels in the lungs or pulmonary arteries (i.e.: pulmonary vasodilators). Examples of vasodilators include Treprostinil and derivatives thereof.

Treprostinil, or 9-deoxy-2′,9-alpha-methano-3-oxa-4,5,6-trinor-3,7-(1′3′-interphenylene)-13,14-dihydro-prostaglandin F1, is a prostacyclin analogue, first described in U.S. Pat. No. 4,306,075. U.S. Pat. No. 5,153,222 describes use of treprostinil for treatment of pulmonary hypertension. Treprostinil is approved for the intravenous as well as subcutaneous route, the latter avoiding septic events associated with continuous intravenous catheters. U.S. Pat. Nos. 6,521,212 and 6,756,033 describe administration of treprostinil by inhalation for treatment of pulmonary hypertension, peripheral vascular disease and other diseases and conditions. U.S. Pat. No. 6,803,386 discloses administration of treprostinil for treating cancer such as lung, liver, brain, pancreatic, kidney, prostate, breast, colon and head-neck cancer. US patent application publication No. 2005/0165111 discloses treprostinil treatment of ischemic lesions. U.S. Pat. No. 7,199,157 discloses that treprostinil treatment improves kidney functions. US patent application publication No. 2005/0282903 discloses treprostinil treatment of neuropathic foot ulcers. U.S. provisional application No. 60/900,320 filed Feb. 9, 2007, discloses treprostinil treatment of pulmonary fibrosis.

The term “acid derivative” is used herein to describe C1-4 alkyl esters and amides, including amides wherein the nitrogen is optionally substituted by one or two C1-4 alkyl groups.

The present invention also encompasses methods of using Treprostinil or its derivatives, or pharmaceutically acceptable salts thereof. In one embodiment, a method uses Treprostinil sodium, currently marketed under the trade name of REMODULIN®. The FDA has approved Treprostinil sodium for the treatment of pulmonary arterial hypertension by injection of dose concentrations of 1.0 mg/mL, 2.5 mg/mL, 5.0 mg/mL and 10.0 mg/mL. The chemical structure formula for Treprostinil sodium is:

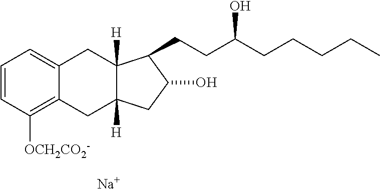

Treprostinil sodium is sometimes designated by the chemical names: (a) [(1R,2R,3aS,9aS)-2,3,3a,4,9,9a-hexahydro-2-hydroxy-1-[(3S)-3-hydroxyoctyl]-1H-benz[f]inden-5-yl]oxy]acetic acid; or (b) 9-deoxy-2′,9-□-methano-3-oxa-4,5,6-trinor-3,7-(1′,3′-interphenylene)-13,14-dihydro-prostaglandin F₁. Treprostinil sodium is also known as: UT-15; LRX-15; 15AU81; UNIPROST™; BW A15AU; and U-62,840. The molecular weight of Treprostinil sodium is 390.52, and its empirical formula is C₂₃H₃₄O₅.

In certain embodiments, treprostinil can be administered in combination with one or more additional active agents. In some embodiments, such one or more additional active agents can be also administered together with treprostinil using a metered dose inhaler. Yet in some embodiments, such one or more additional active agents can be administered separately from treprostinil. Particular additional active agents that can be administered in combination with treprostinil may depend on a particular disease or condition for treatment or prevention of which treprostinil is administered. In some cases, the additional active agent can be a cardiovascular agent such as a calcium channel blocker, a phosphodiesterase inhibitor, an endothelial antagonist, or an antiplatelet agent.

The present invention extends to methods of using physiologically acceptable salts of Treprostinil, as well as non-physiologically acceptable salts of Treprostinil that may be used in the preparation of the pharmacologically active compounds of the invention.

The term “pharmaceutically acceptable salt” refers to a salt of Treprostinil with an inorganic base, organic base, inorganic acid, organic acid, or basic or acidic amino acid. Salts of inorganic bases can be, for example, salts of alkali metals such as sodium or potassium; alkaline earth metals such as calcium and magnesium or aluminum; and ammonia. Salts of organic bases can be, for example, salts trimethylamine, triethylamine, pyridine, picoline, ethanolamine, diethanolamine, and triethanolamine. Salts of inorganic acids can be, for example, salts of hydrochloric acid, hydroboric acid, nitric acid, sulfuric acid, and phosphoric acid. Salts of organic acids can be, for example, salts of formic acid, acetic acid, trifluoroacetic acid, fumaric acid, oxalic acid, lactic acid, tartaric acid, maleic acid, citric acid, succinic acid, malic acid, methanesulfonic acid, benzenesulfonic acid, and p-toluenesulfonic acid. Salts of basic amino acids can be, for example, salts of arginine, lysine and ornithine. Salts of acidic amino acids can include, for example, salts of aspartic acid and glutamic acid. Quaternary ammonium salts can be formed, for example, by reaction with lower alkyl halides, such as methyl, ethyl, propyl, and butyl chlorides, bromides, and iodides, with dialkyl sulphates, with long chain halides, such as decyl, lauryl, myristyl, and stearyl chlorides, bromides, and iodides, and with aralkyl halides, such as benzyl and phenethyl bromides.

Preferred pharmaceutically acceptable salts are disclosed, for example, in US patent application publication No. 20050085540. Physiologically acceptable salts of treprostinil include salts derived from bases. Base salts include ammonium salts (such as quaternary ammonium salts), alkali metal salts such as those of sodium and potassium, alkaline earth metal salts such as those of calcium and magnesium, salts with organic bases such as dicyclohexylamine and N-methyl-D-glucamine, and salts with amino acids such as arginine and lysine. The term “acid derivative” is used herein to describe C1-4 alkyl esters and amides, including amides in which the nitrogen is optionally substituted with one or two C1-4 alkyl groups.

The amount of treprostinil or its derivative, or a pharmaceutically acceptable salt thereof, that is required in a medication or diagnostic aid according to the invention to achieve the desired effect will depend on a number of factors, such as the specific application, the nature of the particular compound used, the mode of administration, the concentration of the compound used, and the weight and condition of the patient. A concentration of the treprostinil or pharmaceutically acceptable salt thereof in the solution ranges from about 500 pg/ml to about 2500 pg/ml, wherein the metered dose inhaler delivers a single dose in a single actuation of treprostinil or pharmaceutically acceptable salt thereof from 30 pg to 90 pg of treprostinil or the pharmaceutically acceptable salt thereof. Described herein is a method for delivering to a subject in need, such as a human, a therapeutically effective amount of treprostinil, which comprises administering to the subject a formulation comprising a therapeutically effective amount of treprostinil, its derivative, or a pharmaceutically salt acceptable from the same, using a metered dose inhaler. Treprostinil can be administered via a metered dose inhaler to a subject suffering from a condition or disease, which can be treated with treprostinil, such as asthma, pulmonary hypertension, peripheral venous insufficiency, or pulmonary fibrosis. In some aspects, an effective amount of Treprostinil is an amount sufficient to reduce KL-6 expression to physiologically acceptable levels. In some aspects, the effective amount is an amount sufficient to result in increased lung function, which may be indicated by an increase in forced vital capacity (FVC).

Treprostinil can be administered by inhalation, which in the present context refers to the delivery of the active ingredient or a combination of active ingredients through a respiratory passage, wherein the subject in need of the active ingredient(s) through the subject's airways, such as the subject's nose or mouth.

A metered dose inhaler in the present context means a device capable of delivering a metered or bolus dose of respiratory drug, such as treprostinil, to the lungs. One example of the inhalation device can be a pressurized metered dose inhaler, a device which produces the aerosol clouds for inhalation from solutions and/or suspensions of respiratory drugs in chlorofluorocarbon (CFC) and/or hydrofluoroalkane (HFA) solutions.

The inhalation device can be also a dry powder inhaler. In such case, the respiratory drug is inhaled in solid formulation, usually in the form of a powder with particle size less than 10 micrometers in diameter or less than 5 micrometers in diameter.

The metered dose inhaler can be a soft mist inhaler (SMI), in which the aerosol cloud containing a respiratory drug can be generated by passing a solution containing the respiratory drug through a nozzle or series of nozzles. The aerosol generation can be achieved in SMI, for example, by mechanical, electromechanical or thermomechanical process. Examples of soft mist inhalers include the Respimat® Inhaler (Boeringer Ingelheim GmbH), the AERx® Inhaler (Aradigm Corp.), the Mystic™ Inhaler (Ventaira Pharmaceuticals, Inc) and the Aira™ Inhaler (Chrysalis Technologies Incorporated). For a review of soft mist inhaler technology, see e.g. M. Hindle, The Drug Delivery Companies Report, Autumn/Winter 2004, pp. 31-34. The aerosol for SMI can be generated from a solution of the respiratory drug further containing pharmaceutically acceptable excipients. In the present case, the respiratory drug is treprostinil, its derivative or a pharmaceutically acceptable salt thereof, which can be formulated in SMI is as a solution. The solution can be, for example, a solution of treprostinil in water, ethanol or a mixture thereof. Preferably, the diameter of the treprostinil-containing aerosol particles is less than about 10 microns, or less than about 5 microns, or less than about 4 microns.

Treprostinil concentration in an aerosolable formulation, such as a solution, used in a metered dose inhaler can range from about 500 μg/ml to about 2500 μg/ml, or from about 800 μg/ml to about 2200 μg/ml, or from about 1000 μg/ml to about 2000 μg/ml.

The dose of treprostinil that can be administered using a metered dose inhaler in a single event can be from about 15 μg to about 100 μg or from about 15 μg to about 90 μg or from about 30 μg to about 90 μg or from about 30 μg to about 60 μg.

Administering of treprostinil in a single event can be carried out in a limited number of breaths by a patient. For example, treprostinil can be administered in 20 breaths or less, or in 10 breaths or less, or than 5 breaths or less. Preferably, treprostinil is administered in 3, 2 or 1 breaths.

The total time of a single administering event can be less than 5 minutes, or less than 1 minute, or less than 30 seconds.

Treprostinil can be administered a single time per day or several times per day.

In some embodiments, the method of treatment of pulmonary hypertension can further comprise administering at least one supplementary agent selected from the group consisting of sildenafil, tadalafil, calcium channel blockers (diltiazem, amlodipine, nifedipine), bosentan, sitaxsentan, ambrisentan, and pharmaceutically acceptable salts thereof. In some embodiments, the supplementary agents can be included in the treprostinil formulation and, thus, can be administered simultaneously with treprostinil using a metered dose inhaler. In some embodiments, the supplementary agents can be administered separately from treprostinil. In some embodiments, the application of intravenous prostacyclin (flolan), intravenous iloprost or intravenous or subcutaneous treprostinil can be administered in addition to treprostinil administered via inhalation using a metered dose inhaler.

### Methods of Treatment

In one aspect, a method of treating a subject having increased expression of KL-6 and suffering from lung disease or lung dysfunction is disclosed. In yet another aspect, a method of treating lung disease or lung dysfunction in a subject suffering therefrom and having an increased expression of KL-6 is disclosed. In yet another aspect, a method of reducing KL-6 expression in a subject suffering from lung disease or lung dysfunction. In yet another aspect, a method of treating lung disease or lung dysfunction in a subject suffering therefrom and having previously been determined as having increased expression of KL-6 is disclosed.

In some aspects, the methods comprise, consist of, or consist essentially of administering an effective amount of a vasodilator to the subject. In some aspects, the vasodilator comprises, consists of, or consists essentially of Treprostinil or a salt thereof. In some aspects, the effective amount of the vasodilator is an amount sufficient to reduce KL-6 expression. In some aspects, the effective amount of the vasodilator is an amount sufficient to decease KL-6 expression to below 600 U/ml, or below 650 U/ml, or below 700 U/ml or below 750 U/ml. In some aspects, the Treprostinil or salt thereof is administered by inhalation at a concentration of about 500 μg/ml to about 2500 μg/ml. In some aspects, the effective amount of the vasodilator is an amount sufficient to reduce KL-6 expression and increase forced vital capacity above 2.05 L, or 2.1 L, or 2.15 L, or 2.2 L. In some aspects, the effective amount is between 5-500 μg inhaled Treprostinil per day. In some aspects, administration of the effective amount of the vasodilator results in increased force vital capacity in the subject.

In some aspects, the method further comprise, consist of, or consist essentially of the step of detecting the level of KL-6 expression prior to administration of the vasodilator. In some aspects, the method further comprise, consist of, or consist essentially of the step of detecting the level of KL-6 expression following administration of the vasodilator.

In some aspects, the lung disease comprises, consists of, or consists essentially of pulmonary hypertension associated with interstitial lung disease (PH-ILD).

In some aspects, the patient exhibits a baseline expression of KL-6 that is greater than 600 U/ml, or greater than 650 U/ml, or greater than 700 U/ml, or greater than 750 U/ml, or greater than 800 U/ml prior to administration of the vasodilator. In some aspects, the subject exhibits a baseline forced vital capacity of less than 2.20 L, or less than 2.15 L, or less than 2.10 L prior to administration of the vasodilator.

In some aspects, the vasodilator is administered by intravenous administration, intramuscular administration, oral administration, or inhalation.

In some aspects, the subject is a mammalian subject. In yet another aspect, the subject is a human subject. In some aspects, the subject has mean pulmonary arterial pressure (mPAP)≥25 mmHg and a pulmonary arterial wedge pressure (PAWP)≤15 mmHg as confirmed by a baseline right heart catheterization (RHC). In yet another aspect, the subject is suffering from interstitial pneumonia.

### EXAMPLES

These examples are provided for illustrative purposes only and not to limit the scope of the claims provided herein.

### Example 1

### Introduction

Treprostinil is a chemically stable analogue of prostacyclin, which promotes vasodilation of pulmonary and systemic arterial vascular beds,⁶ inhibits both platelet aggregation and pulmonary artery smooth muscle cell proliferation, and reverses pulmonary artery remodeling. Inhaled treprostinil was previously shown to improve hemodynamics and exercise capacity after 12 weeks of therapy in Japanese patients with PAH.⁷ The recently published the INCREASE trial was for the first time able to show a significant, positive effect of inhaled treprostinil in a population of patients with PH-ILD.⁸ The INCREASE trial demonstrated that inhaled treprostinil can improve exercise capacity and decrease clinical worsening without any significant safety issues. However, the efficacy of inhaled treprostinil on hemodynamic and PK parameters was not assessed in the INCREASE trial. Therefore, the objective of this trial was to evaluate efficacy on hemodynamic parameters and exercise capacity, safety and PK of inhaled treprostinil in Japanese patients with PH-ILD.

### Materials and Methods

Trial and Design: This trial was a multicenter, non-randomized, open-label, single-arm, 52-week trial of patients with PH-ILD. Inhaled treprostinil (0.6 mg/mL) was administered by means of an ultrasonic, pulsed-delivery nebulizer at 6 μg/breath. Patients initiated treprostinil inhalation at three breaths, four times daily. If clinically tolerated, the dose was gradually increased to a maximum of 12 breaths four times daily in one-breath increments with a minimum interval of 3 days. This trial was conducted in accordance with the Declaration of Helsinki and Good Clinical Practice guidelines and approved by the Institutional Review Board of Chiba University Hospital as well as all other participating trial site. Seventeen facilities participated in this trial, and patients were enrolled in 9 facilities. All Patients gave written informed consent prior to enrollment.

Patients: Eligible subjects were Japanese patients aged 18 to 80 years who had ILD diagnosed on the basis of evidence of diffuse parenchymal lung disease on computed tomography of the chest performed within 6 months before treatment. Patients could be in any World Health Organization (WHO) functional class: I-IV. Patients were required to have a baseline 6-minute walking distance (6MWD)≥100 m, and to have mean pulmonary arterial pressure (mPAP)≥25 mmHg with pulmonary arterial wedge pressure (PAWP)≤15 mmHg, confirmed by a baseline right heart catheterization (RHC). Patients were also required to have pulmonary vascular resistance (PVR) >3 Wood units, as in the INCREASE trial. Patients were enrolled if their physician determined that their degree of PH was stable, by comparing PVR measured by RHC or transtricuspid pressure gradient (TRPG) measured by echocardiography at baseline, with measurements taken at least 14 days but within 60 days prior to the baseline readings. Patients were excluded if corticosteroid (oral, intravenously, subcutaneously, intra-articularly or epidurally) had been administered at a daily dose of >20 mg of prednisolone, or they had received oral immunosuppressive drugs within the past 30 days, counted from baseline. Patients with connective tissue disease were also required to have a baseline forced vital capacity (FVC) percent predicted <70%. Patients receiving drug treatment (i.e., pirfenidone or nintedanib) for their underlying lung disease were required to have been receiving a stable dose for at least 30 days before enrollment. Concomitant use of PAH-specific therapy within 60 days prior to enrollment was not permitted.

Endpoint: The primary endpoints of this trial were the change in pulmonary vascular resistance index (PVRI) and peak 6MWD from baseline to week 16. Hemodynamic measurements by RHC were performed at baseline and week 16, pre-inhalation, 15 min after inhalation, and 30 min after inhalation. Cardiac output (CO) was measured by either the thermodilution or indirect Fick method. The same method was used for each patient both at baseline and at week 16. The calculation method details on hemodynamic parameters are provided below. Regarding peak 6MWD, the 6-minute walk test was performed at baseline and at weeks 4, 8, 12, 16, 24, 32, 40 and 52, or at the time of early discontinuation of treprostinil. Peak 6MWD was measured within 10-60 min after inhaled treprostinil administration. Secondary efficacy endpoints included changes in other hemodynamic parameters, N-terminal fragment of brain natriuretic peptide (NT-proBNP) levels, WHO functional class.

Safety endpoints included adverse events (AEs), routine laboratory parameters measured in a clinical laboratory, supplemental oxygen requirement at rest, pulmonary function tests, arterial blood gas analysis.

Calculation Methods: Cardiac index was derived by correction of CO with BSA: Cardiac index=CO/BSA. PVR and PVRI were calculated from trans-pulmonary pressure gradient and pulmonary blood flow: PVR=(mPAP−PAWP)/CO, and PVRI=(mPAP−PAWP)/Cardiac index, respectively. Abbreviations: BSA, body surface area; CO, cardiac output; mPAP, mean pulmonary arterial pressure; PAWP, pulmonary arterial wedge pressure; PVR, pulmonary vascular resistance; PVRI, pulmonary vascular resistance index.

Pharmacokinetic Data: For analysis of inhaled treprostinil PK in the Japanese patients in this trial, blood samples were collected at baseline and pre-inhalation, and up to 240 minutes after inhalation in week 16. Plasma treprostinil concentrations were measured using a liquid chromatography tandem mass spectrometry assay. PK parameters, determined from the plasma concentration versus time data for treprostinil, included the maximum plasma concentration (Cₘₐₓ), time to Cₘₐₓ (Tₘₐₓ), elimination half time (T_{1/2}), area under the plasma concentration-time curve (AUC), and AUC from time zero to infinity (AUCinf).

Secondary Endpoints—Efficacy: Secondary efficacy endpoints included changes in other hemodynamic parameters, trough 6-minute walking distance (6MWD), N-terminal fragment of brain natriuretic peptide (NT-proBNP) levels, World Health Organization (WHO) functional class, quality of life measured by the St George's Respiratory Questionnaire (SGRQ) and the five-level version of the EuroQol five-dimensional descriptive system (EQ-5D-5L), distance saturation product (DSP), and the time to clinical worsening. Trough 6MWD was measured at least after 4 hours post-inhalation.

Secondary Endpoints—Safety: Safety endpoints included adverse events, routine laboratory parameters measured in a clinical laboratory, electrocardiograms, vital signs (blood pressure, pulse), Oxygenation (percutaneous arterial oxygen saturation (SpO₂) and the amount of supplemental oxygen at rest), pulmonary function tests, arterial blood gas analysis, chest X-ray, body weight, hospitalization due to a cardiopulmonary indication, and cardiac ultrasound examination.

Pharmacokinetics (PK) Parameters: PK parameters, determined from the plasma concentration versus time data for treprostinil, included the maximum plasma concentration (Cₘₐₓ), plasma concentration at measurable final time point (Cₗₐₛₜ), time to Cₘₐₓ (Tₘₐₓ), elimination half time (T_{1/2}), time of the last measurable plasma concentration (Tₗₐₛₜ), area under the plasma concentration-time curve (AUC) from time zero to last measurable concentration sampling time (AUCₗₐₛₜ), AUC from time zero to infinity (AUCinf), percentage of AUCinf based on extrapolation (AUC_{Extrap}), elimination rate constant (λ_{Z}), mean residence time (MRT), apparent total body clearance (CL/F) and apparent volume of distribution during the terminal phase (V_{Z}/F).

Statistical Analysis: Efficacy, safety and PK analysis sets included all patients who received one dose of treprostinil inhalation and had any single post-baseline efficacy, safety and PK datum, respectively. The lower PVRI measured at 15 or 30 minutes after inhalation was taken as the designated “best” PVRI at week 16. The criterion for demonstrating the efficacy of PVRI was defined as the upper limit of the two-sided 95% CI of the mean change in PVRI from baseline to week 16 being below the value +7%, which was the change observed in the placebo group from baseline to week 16 in the BPHIT trial (bosentan in PH-ILD treatment trial).⁹ In the primary analysis of PVRI, the percent change in PVRI was set to +7% for patients who had no PVRI at week 16 due to death or worsening of ILD or PH. Last Observation Carried Forward (LOCF) or Baseline Observation Carried Forward (BOCF) was used for imputation for all other patients who did not have PVRI measured at week 16. Regarding 6MWD, Applicant evaluated similarity between the results of this trial and the INCREASE trial. Similarity of the trials was evaluated by comparing the median change in 6MWD from baseline to week 16 and its two-sided 95% CI, obtained from both trials. In the primary analysis of the peak 6MWD, the change in 6MWD was set to 0 m for patients who had no 6MWD measurement at week 16 due to death or worsening of ILD or PH. LOCF or BOCF was used for imputation for all other patients who did not have a 6MWD measurement at week 16.

AEs were summarized by preferred term using the Medical Dictionary for Regulatory Activities Version 26.0. All statistical analysis was performed using SAS (version 9.4).

### Results

Patients: Patient demographic data are provided in Table 1. A total of 20 patients were enrolled, with a mean age of 65.6±13.0 years. The most common diagnosis was idiopathic interstitial pneumonia (60.0%), and the mean time since the diagnosis of PH was 0.7±1.6 years. At baseline, the mean PVR was 5.9±3.1 Wood units and the mean 6MWD was 313.9±107.0 m. WHO functional classes at baseline were mostly class III (85.0%). Eighteen patients completed the 16-week main treatment period and all patients entered the long-term treatment period; 16 patients continued to week 52. During the 16-week period, the median maximum dose of treprostinil was 12.0 breaths/session.

TABLE 1
Characteristics of the patients at baseline [n (%)] (n = 20)

| | | |
|---|---|---|
| Female | 7 | (35.0) |
| Age (years) | | |

| | |
|---|---|
| Mean ± SD | 65.6 ± 13.0 |

Time since PH diagnosis (years)

| | |
|---|---|
| Mean ± SD | 0.7 ± 1.6 |

| | | |
|---|---|---|
| Cause of lung disease | | |
| Idiopathic interstitial pneumonia | 12 | (60.0) |
| Connective tissue disease | 5 | (25.0) |
| Combined pulmonary fibrosis and emphysema | 2 | (10.0) |
| Chronic hypersensitivity pneumonitis | 1 | (5.0) |
| Idiopathic interstitial pneumonia subcategory | | |
| Idiopathic pulmonary fibrosis | 7 | (35.0) |
| Respiratory bronchiolitis associated | 1 | (5.0) |
| with interstitial lung disease | | |
| Idiopathic pulmonary upper lobe fibrosis | 1 | (5.0) |
| Unclassified idiopathic interstitial pneumonia | 3 | (15.0) |
| Use of supplemental oxygen | 15 | (75.0) |
| Background antifibrotic therapy | | |
| None | 10 | (50.0) |
| Pirfenidone only | 4 | (20.0) |
| Nintedanib only | 6 | (30.0) |
| Baseline WHO functional class | | |
| I | 1 | (5.0) |
| II | 2 | (10.0) |
| III | 17 | (85.0) |
| Baseline PVR (Wood units) | | |

| | |
|---|---|
| Mean ± SD | 5.9 ± 3.1 |

Baseline PVRI (Wood units · m²)

| | |
|---|---|
| Mean ± SD | 9.7 ± 5.0 |

Baseline 6MWD (m)

| | |
|---|---|
| Mean ± SD | 313.9 ± 107.0 |

Abbreviations: ILD, interstitial lung disease; PH, pulmonary hypertension; PVR, pulmonary vascular resistance; PVRI, pulmonary vascular resistance index; 6MWD, 6-minute walking distance; WHO, World Health Organization.

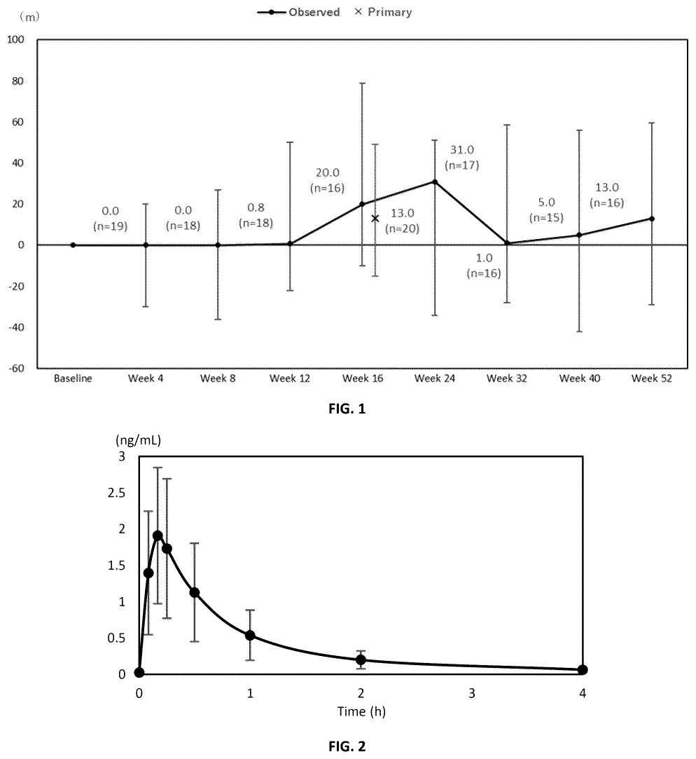

Efficacy: Hemodynamic parameters, including the primary endpoint of PVRI, at baseline and at week 16 are summarized in Table 2. The primary analysis for PVRI showed that the mean (±SD) of the best change from baseline at week 16 was −40.1±27.7% (95% CL: −53.1 to −27.2). In addition, improvement from baseline was observed in other hemodynamic parameters at the best of week 16: mean (±SD) PVR and mPAP decreased by −2.4±2.3 Wood units and −7.6±7.2 mmHg, respectively. The primary analysis for peak 6MWD showed that the median change from baseline to week 16 was ±13.0 m (95% CI: −15.0 to 49.0). For patients with available data (observed), the median change from baseline at week 16 was ±20.0 m. This improvement was also observed at week 52 (FIG. 1). The mean (±SD) changes from baseline in NT-proBNP level, using LOCF or BOCF for imputation, were −36.9±218.4 pg/mL (95% CI: −139.1 to 65.3) and −30.8±466.1 pg/mL (95% CI: −248.9 to 187.3) at Week 16 and 52, respectively. Other main secondary endpoints results at baseline, week 16 and week 52 are shown in Table 3 and Table 5.

Table 4 shows the effects of Treprostinil administration on KL-6 expression. As demonstrated in Table 4, various doses of Treprostinil therapy effectively reduce KL-6 expression in subjects.

TABLE 2
Hemodynamic Parameters (n = 20)

| | | | | | | Change from | % Change from |
|---|---|---|---|---|---|---|---|
| | | | | | | baseline to Week 16 | baseline to Week 16 |
| | | Week 16 | | | | Post-inhalation | Post-inhalation |
| | | | Post-inhalation | | | Best | Best |
| | Baseline | Pre-inhalation | 15 min | 30 min | Best | Mean ± SD | Mean ± SD |
| | Mean ± SD | Mean ± SD | Mean ± SD | Mean ± SD | Mean ± SD | (95% CI) | (95% CI) |
| PVRI | 9.7 ± 5.0 | 9.3 ± 5.9 | 6.0 ± 3.3 | 6.0 ± 2.9 | 5.6 ± 3.2 | −4.1 ± 3.7 | −40.1 ± 27.7 |
| (Wood units · m²) | | | | | | (−5.8 to −2.4) | (−53.1 to −27.2) |
| PVR | 5.9 ± 3.1 | 5.6 ± 3.6 | 3.7 ± 2.2 | 3.7 ± 2.0 | 3.5 ± 2.1 | −2.4 ± 2.3 | 40.2 ± 27.7 |
| (Wood units) | | | | | | (−3.5 to −1.3) | (−53.1 to −27.2) |
| mPAP | 33.3 ± 7.1 | 33.7 ± 9.9 | 26.7 ± 6.7 | 26.3 ± 7.2 | 25.7 ± 6.7 | −7.6 ± 7.2 | −21.6 ± 18.9 |
| (mmHg) | | | | | | (−11.0 to −4.2) | (−30.5 to −12.8) |
| CO^{a} | 4.4 ± 0.8 | 4.3 ± 0.9 | 4.8 ± 1.0 | 4.8 ± 1.0 | 4.9 ± 1.0 | 0.6 ± 0.8 | 13.4 ± 17.2 |
| (L/min) | | | | | | (0.2 to 0.9) | (5.4 to 21.4) |
| Cardiac index | 2.6 ± 0.5 | 2.6 ± 0.5 | 2.9 ± 0.6 | 2.9 ± 0.6 | 3.0 ± 0.6 | 0.3 ± 0.5 | 13.2 ± 17.1 |
| (L/min/m²) | | | | | | (0.1 to 0.5) | (5.3 to 21.2) |
| SVR | 23.1 ± 6.8 | 23.5 ± 7.1 | 20.1 ± 6.2 | 20.4 ± 6.2 | 19.7 ± 6.3 | −3.4 ± 4.0 | −14.6 ± 14.2 |
| (Wood units) | | | | | | (−5.3 to −1.6) | (−21.2 to −7.9) |
| SVRI | 38.3 ± 10.8 | 38.9 ± 11.1 | 33.2 ± 8.6 | 33.5 ± 8.5 | 32.4 ± 8.6 | −5.9 ± 6.5 | −14.5 ± 14.0 |
| (Wood units · m²) | | | | | | (−9.0 to −2.9) | (−21.0 to −7.9) |
| PAWP | 9.1 ± 3.3 | 10.6 ± 3.6 | 10.2 ± 4.5 | 9.7 ± 4.0 | 9.4 ± 4.0 | 0.4 ± 3.2 | 8.0 ± 42.9 |
| (mmHg) | | | | | | (−1.1 to 1.9) | (−12.1 to 28.1) |
| mRAP | 4.4 ± 3.4 | 4.6 ± 3.3 | 3.3 ± 2.9 | 3.4 ± 3.5 | 2.7 ± 2.5 | −1.7 ± 2.6 | −36.4 ± 45.3^{b} |
| (mmHg) | | | | | | (−2.9 to −0.5) | (−59.6 to −13.1) |
| SvO₂ | 68.4 ± 6.2 | 68.1 ± 7.8 | 67.7 ± 6.6 | 66.9 ± 5.8 | 68.0 ± 6.5 | −0.4 ± 4.4 | −0.4 ± 6.5 |
| (%) | | | | | | (−2.5 to 1.7) | (−3.5 to 2.7) |

In the primary analysis of PVRI, the change of PVRI was set to +7% for patients who had no PVRI at week 16 due to death or worsening of ILD or PH. LOCF was used for imputation for all other patients who did not have PVRI at week 16.
^{a}CO was measured by the thermodilution (n = 17) or indirect Fick method (n = 3).
^{b}n = 17.
Abbreviations: CI, confidence interval; CO, cardiac output; mPAP, mean pulmonary arterial pressure; mRAP, mean right atrial pressure; PAWP, pulmonary arterial wedge pressure; PVR, pulmonary vascular resistance; PVRI, pulmonary vascular resistance index; SvO₂, mixed venous oxygen saturation; SVR, systemic vascular resistance; SVRI, systemic vascular resistance index.

TABLE 3
Other secondary endpoints

| | | | | Change | Change |
|---|---|---|---|---|---|
| | | | | from baseline | from baseline |
| | Baseline | Week 16 | Week 52 | to Week 16 | to Week 52 |
| | Mean ± SD | Mean ± SD | Mean ± SD | Mean ± SD | Mean ± SD |
| KL-6 (U/mL) | 785.3 ± 483.4 | 786.3 ± 552.0 | 593.8 ± 313.4 | −53.6 ± 325.9 | −216.6 ± 245.4 |
| | (n = 20) | (n = 18) | (n = 16) | (n = 18) | (n = 16) |
| FVC (L) | 2.04 ± 0.72 | 2.21 ± 0.75 | 2.13 ± 0.73 | 0.10 ± 0.17 | 0.03 ± 0.31 |
| | (n = 20) | (n = 17) | (n = 16) | (n = 17) | (n = 16) |
| FVC (% predicted) | 62.7 ± 19.9 | 66.3 ± 22.1 | 64.1 ± 21.5 | 3.3 ± 5.5 | 1.9 ± 9.7 |
| | (n = 20) | (n = 17) | (n = 16) | (n = 17) | (n = 16) |
| PaO₂ (mmHg) | 66.6 ± 22.5 | 67.5 ± 28.9 | 69.1 ± 18.1 | 0.9 ± 24.6 | 1.9 ± 15.5 |
| | (n = 20) | (n = 20) | (n = 18) | (n = 20) | (n = 18) |
| PaCO₂ (mmHg) | 44.2 ± 8.7 | 45.2 ± 11.0 | 45.2 ± 10.3 | 1.0 ± 5.2 | 1.9 ± 5.5 |
| | (n = 20) | (n = 20) | (n = 18) | (n = 20) | (n = 18) |
| The amount of | 1.3 ± 1.3 | 1.6 ± 1.2 | 1.8 ± 1.5 | 0.3 ± 1.0 | 0.5 ± 0.9 |
| supplemental oxygen | (n = 20) | (n = 18) | (n = 16) | (n = 18) | (n = 16) |
| required at rest | | | | | |
| (L/min) | | | | | |

Abbreviations: FVC, forced vital capacity; KL-6, Krebs von den Lungen-6; PaCO₂, arterial partial pressure of carbon dioxide; PaO₂, arterial partial pressure of oxygen.

TABLE 4
Treprostinil Dose vs. KL-6 Expression.

| | | Week 16 (n = 18) | | | Week 52(n = 16) | | |
|---|---|---|---|---|---|---|---|
| | | | | Change from | | | Change from |
| | Baseline | | | Baseline to | | | Baseline to |
| | KL-6 | Breaths per | KL-6 | Week 16 | Breaths per | KL-6 | Week 52 |
| Subject | (U/mL) | session | (U/mL) | (U/mL) | session | (U/mL) | (U/mL) |
| A | 1170 | 12 | 1150 | −20 | 12 | 1020 | −150 |
| B | 262 | 10 | 247 | −15 | 10 | 270 | 8 |
| C | 1450 | 12 | 2470 | 1020 | | Discontinued | |
| D | 1210 | 12 | 1090 | −120 | 12 | 1090 | −120 |
| E | 1050 | 12 | 1110 | 60 | 12 | 875 | −175 |
| F | 287 | 9 | 276 | −11 | 9 | 247 | −40 |
| G | 246 | . | Discontinued | | | Discontinued | |
| H | 1840 | 12 | 1340 | −500 | 12 | 1060 | −780 |
| I | 291 | 12 | 203 | −88 | 12 | 244 | −47 |
| J | 558 | 7 | 399 | −159 | 8 | 360 | −198 |
| K | 410 | 12 | 401 | −9 | 12 | 392 | −18 |
| L | 369 | 12 | 481 | 112 | 12 | 425 | 56 |
| M | 703 | 12 | 641 | −62 | | Discontinued | |
| N | 1190 | 12 | 1160 | −30 | 12 | 939 | −251 |
| O | 540 | 12 | 545 | 5 | 12 | 292 | −248 |
| P | 735 | 12 | 508 | −227 | 12 | 567 | −168 |
| Q | 493 | 12 | 437 | −56 | 11 | 382 | −111 |
| R | 1450 | 12 | 832 | −618 | 12 | 743 | −707 |
| S | 1110 | 12 | 864 | −246 | 12 | 594 | −516 |
| T | 341 | | Discontinued | | | Discontinued | |

TABLE 5
Summary of WHO functional class [n (%))]

| Baseline (n = 20) | | |
|---|---|---|
| I | 1 | (5.0) |
| II | 2 | (10.0) |
| III | 17 | (85.0) |
| Week 16 (n = 19) | | |
| I | 1 | (5.3) |
| II | 4 | (21.1) |
| III | 13 | (68.4) |
| IV | 1 | (5.3) |
| Shift from baseline to Week 16 (n = 19) | | |
| I −> I | 1 | (5.3) |
| II −> II | 1 | (5.3) |
| III −> II | 3 | (15.8) |
| III −> III | 13 | (68.4) |
| III −> IV | 1 | (5.3) |
| Week 52 (n = 16) | | |
| I | 1 | (6.3) |
| II | 3 | (18.8) |
| III | 12 | (75.0) |
| Shift from baseline to Week 52 (n = 16) | | |
| I −> I | 1 | (6.3) |
| II −> II | 1 | (6.3) |
| III −> II | 2 | (12.5) |
| III −> III | 12 | (75.0) |

Abbreviation: WHO, World Health Organization.

Safety: Inhaled treprostinil therapy four times daily was well tolerated in Japanese patients with PH-ILD.

AEs reported over 52 weeks are summarized in Table 6. All 20 patients experienced at least one AE, and one AE related to treprostinil, during the 52-week period. The most frequently reported AEs related to treprostinil were cough (50.0%), malaise (10.0%) and blood pressure decreased (10.0%). Eight patients experienced serious AEs: pneumonia (15.0%); pneumonia bacterial, acute myocardial infarction and right ventricular failure (10.0%); and cataract, cardiac failure congestive, and bronchiolitis (5.0%). Although the serious pneumonia that occurred in one patient was considered to be causally related, the patient recovered after discontinuation of treprostinil. Four patients discontinued the trial due to AEs: dyspnoea (10.0%); and cough, cardiac failure congestive, and pneumonia (5.0%). AEs that led to discontinuation of the treprostinil were dyspnea, cough, and pneumonia (5.0%), all of which resolved after discontinuation of the treprostinil.

There were no clinically relevant changes found in other safety endpoints during the 52-week period.

TABLE 6
Summary of AEs Over 52 Weeks [n (%)] (n = 20)

| Patients with AEs | | |
|---|---|---|
| Total patients with at least one AE | 20 | (100.0) |
| Total patients with at least one AE | 14 | (70.0) |
| related to treprostinil | | |
| Deaths | 0 | (0.0) |
| Total patients with at least one serious AE | 8 | (40.0) |
| Total patients with at least one serious | 1 | (5.0) |
| AE related to treprostinil | | |
| Total patients with at least one AE | 4 | (20.0) |
| leading to withdrawal of treprostinil | | |
| AEs related to treprostinil | | |
| Cough | 10 | (50.0) |
| Malaise | 2 | (10.0) |
| Blood pressure decreased | 2 | (10.0) |
| Pneumonia | 1 | (5.0) |
| Dysgeusia | 1 | (5.0) |
| Dyspnoea | 1 | (5.0) |
| Throat irritation | 1 | (5.0) |
| Flank pain | 1 | (5.0) |
| Myalgia | 1 | (5.0) |
| Pain in jaw | 1 | (5.0) |
| Feeling abnormal | 1 | (5.0) |
| Oedema | 1 | (5.0) |
| Sensation of foreign body | 1 | (5.0) |

Abbreviation: AE, adverse event.

PK: The PK of inhaled treprostinil in the plasma was examined in 15 patients, at the maximum dose of 12 breaths (72 μg)/session. Treprostinil was absorbed with a mean Tₘₐₓ of 0.18 h, eliminated with T_{1/2} of 0.95 h and remained detectable in the plasma until approximately 4 h after inhalation, at the maximum dose of 12 breaths (72 μg) four times daily (FIG. 2). The mean values of Cₘₐₓ, AUCₗₐₛₜ and AUCinf were 1.96 ng/mL, 1.71 h-ng/mL and 1.79 h-ng/mL, respectively. Other PK parameters are shown in Table 7.

TABLE 7
Pharmacokinetic Parameters of 72 μg of Inhaled Treprostinil

| | n | Mean ± SD |
|---|---|---|
| Cₘₐₓ (ng/mL) | 15 | 1.96 ± 0.97 |
| Cₗₐₛₜ (ng/mL) | 15 | 0.06 ± 0.03 |
| Tₘₐₓ (h) | 15 | 0.18 ± 0.05 |
| T_{1/2} (h) | 15 | 0.95 ± 0.16 |
| Tₗₐₛₜ (h) | 15 | 3.96 ± 0.07 |
| AUCₗₐₛₜ (h · ng/mL) | 15 | 1.71 ± 0.93 |
| AUC_{inf} (h · ng /mL) | 15 | 1.79 ± 0.95 |
| AUC_{Extrap} (%) | 15 | 5.69 ± 3.54 |
| λ_{Z} (/h) | 15 | 0.74 ± 0.11 |
| MRT (h) | 15 | 1.16 ± 0.27 |
| CL/F (L/h) | 15 | 56.87 ± 39.46 |
| V_{Z}/F (L) | 15 | 82.60 ± 66.22 |

Abbreviations: AUC, area under the plasma concentration-time curve; AUC_{Extrap}, percentage of AUC_{inf} based on extrapolation; AUC_{inf}, AUC from time zero to infinity; AUCₗₐₛₜ, AUC from time zero to last measurable concentration sampling time; CL/F, apparent total body clearance; Cₗₐₛₜ, plasma concentration at measurable final time point; Cₘₐₓ, the maximum plasma concentration; MRT, mean residence time; T_{1/2}, elimination half time; Tₗₐₛₜ, time of the last measurable plasma concentration; Tₘₐₓ, time to _{Cmax}; V_{Z}/F, apparent volume of distribution during the terminal phase; λ_{Z}, elimination rate constant.

### Discussion

This trial is the first to evaluate the efficacy of inhaled treprostinil on hemodynamic parameters and PK in patients with PH-ILD. Considering that PVR has been reported to predict the prognosis of Group 3 patients with ILD,¹⁰⁻¹² Applicant considered PVRI as a surrogate endpoint for death. Additionally, since this trial was a non-randomized, open-label trial, Applicant deemed it appropriate to set PVRI as one of the primary endpoints, an objective evaluation index. On the other hand, since sufficient data cannot be obtained to adjudicate clinically significant improvements in PVRI in PH-ILD, the 6MWD, which was the primary endpoint in the INCREASE trial, was also set as the primary endpoint in this trial.

Regarding hemodynamics, the mean change in PVRI at week 16 in this trial was −40.1±27.7% (95% CI: −53.1 to −27.2), which was a great improvement and the upper limit of the CI was lower than the result of +7% in the placebo group of the BPHIT trial. Applicant confirmed that the predefined success criteria were met for the change in PVRI from baseline to week 16. In addition, the improvements observed in mPAP and NT-proBNP are consistent with the PVRI changes. Regarding exercise capacity, the median change in peak 6MWD and 95% CI of the inhaled treprostinil group in this trial and in the INCREASE trial were +13.0 m (95% CI: −15.0 to 49.0) and +6.0 m (95% CI: 0.0 to 14.0), respectively. The median change in 6MWD from baseline to week 16 in this trial was within the 95% CI obtained in the INCREASE trial, and vice versa. This result suggests that there is similarity between the results of the 6MWD obtained in the two trials. Furthermore, PVRI showed significant improvement beyond the expected −20% when the trial was designed, and the 6MWD also demonstrated an improvement from baseline to week 52.

In this trial, FVC and Krebs von den Lungen-6 (KL-6) were measured continually during the 52-week period. Interestingly, FVC showed an increase compared to baseline throughout the trial period. Similarly, KL-6 showed a decrease throughout the trial period and reached the lowest value at 52 weeks. KL-6 is one of the most useful serum biomarkers in patients with IPF and it has been reported that increases in KL-6 are related to declines in FVC.¹³ The BPHIT trial in patients with PH-ILD demonstrated a decline in FVC with the administration of pulmonary vasodilators.⁹ However, the INCREASE trial reported an increase in FVC.¹⁴ Furthermore, this trial not only observed a similar trend to the INCREASE trial in FVC, but it also confirmed a decrease in KL-6. The reasons for these results are potentially attributable to the antifibrotic properties of treprostinil, but further investigation is required.

The safety profile of inhaled treprostinil observed in this trial was similar to that reported in the INCREASE trial. The most frequently reported AEs related to treprostinil were cough, malaise and blood pressure decreased, which were typical of prostacyclin therapy or associated with the inhaled route of delivery of the drug. Most of the reported AEs related to treprostinil in this trial were non-serious and of mild-to-moderate intensity, excluding pneumonia. There were no deaths. Four patients discontinued the trial due to AEs: dyspnoea (10.0%), and cough, cardiac failure congestive, and pneumonia (5.0%). In addition, arterial blood gas analysis was assessed at baseline, week 16 and week 52, but no significant changes were observed. No significant changes in the amount of supplemental oxygen were observed at baseline, week 16, and week 52, suggesting that inhaled treprostinil did not have a negative impact on oxygenation.

Finally, this is the first trial to investigate the PK of inhaled treprostinil in patients with PH-ILD, with administration of 72 μg. At week 16, the maximum concentration was achieved at 10 minutes post-dose (1.91 ng/mL) with almost complete elimination by 240 minutes post-dose. This result was similar to the findings reported in the trials conducted on Japanese patients with PAH.⁷

The major limitation of this trial is that the trial design was open-label and single-arm. Therefore, subjective parameters such as WHO functional class might be biased, compared to objective measures such as hemodynamic parameters and NT-proBNP levels. In addition, the number of patients enrolled was small and the duration of evaluation in this trial was just 52 weeks. Furthermore, there is a need to identify patient groups in whom inhaled treprostinil is more effective. Finally, efficacy and safety were not compared with a placebo or active control. However, exercise capacity, symptoms and tolerability in this trial showed the same directional changes as results of the INCREASE trial, which was a large-scale, multi-regional, randomized, double-blind, and placebo-controlled trial.

### Conclusions

Applicant demonstrates that inhaled treprostinil is well tolerated and shows an acceptable safety profile in addition to improvements in pulmonary hemodynamics, exercise capacity, and symptoms. This trial shows very consistent results regarding the positive effects of inhaled treprostinil in a population of patients with PH-ILD, and clearly supports the use of KL-6 as a biomarker for diagnosing and treating PH-ILD with treprostinil. Further, the data surprisingly demonstrates that KL-6 is a biomarker for identifying patients who are especially susceptible to PH-ILD treatment with treprostinil. The data demonstrates that KL-6 serves as both a suitable diagnostic for patient selection for treprostinil treatment as well as a biomarker for measuring the efficacy of treprostinil treatment. Since there are no other treatment options available, inhaled treprostinil may also be a valuable therapeutic option for patients with PH-ILD, particularly patients with elevated KL-6 expression.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### EMBODIMENTS

1. A method of treating a subject having increased expression of KL-6 and suffering from lung disease or lung dysfunction comprising administering an effective amount of a vasodilator to the subject.

2. A method of treating lung disease or lung dysfunction in a subject suffering therefrom and having an increased expression of KL-6, comprising administering an effective amount of a vasodilator to the subject.

3. A method of reducing KL-6 expression in a subject suffering from lung disease or lung dysfunction, comprising administering an effective amount of a vasodilator to the subject.

4. A method of treating lung disease or lung dysfunction in a subject suffering therefrom and having previously been determined as having increased expression of KL-6, comprising administering an effective amount of a vasodilator to the subject.

5. The method of any one of embodiments 1-4, wherein the vasodilator comprises Treprostinil or a salt thereof.

6. The method of any one of embodiments 1-5, wherein the effective amount of the vasodilator is an amount sufficient to reduce KL-6 expression.

7. The method of any one of embodiments 1-6, wherein the lung disease comprises pulmonary hypertension associated with interstitial lung disease (PH-ILD).

8. The method of any one of embodiments 1-6, wherein the patient exhibits a baseline expression of KL-6 that is greater than 600 U/ml, or greater than 650 U/ml, or greater than 700 U/ml, or greater than 750 U/ml, or greater than 800 U/ml prior to administration of the vasodilator.

9. The method of embodiment 8, wherein the effective amount of vasodilator is an amount sufficient to decease KL-6 expression to below 600 U/ml, or below 650 U/ml, or below 700 U/ml or below 750 U/ml.

10. The method of any one of embodiments 1-9, wherein the vasodilator is administered by intravenous administration, intramuscular administration, oral administration, or inhalation.

11. The method of any one of embodiments 1-10, wherein the subject is a mammalian subject, optionally, wherein the subject is a human subject.

12. The method of any one of embodiments 5-11, wherein the Treprostinil or salt thereof is administered by inhalation at a concentration of about 500 μg/ml to about 2500 μg/ml.

13. The method of any one of embodiments 1-12, wherein the subject exhibits a baseline forced vital capacity of less than 2.20 L, or less than 2.15 L, or less than 2.10 L prior to administration of the vasodilator.

14. The method of any one of embodiments 6-13, wherein the effective amount of the vasodilator is an amount sufficient to reduce KL-6 expression and increase forced vital capacity above 2.05 L, or 2.1 L, or 2.15 L, or 2.2 L.

15. The method of any one of embodiments 5-14, wherein the effective amount is between 5-500 μg inhaled treprostinil per day.

16. The method of any one of embodiments 1-15, further comprising the step of detecting the level of KL-6 expression prior to administration of the vasodilator.

17. The method of any one of embodiments 1-16, further comprising the step of detecting the level of KL-6 expression following administration of the vasodilator.

18. The method of any one of embodiments 1-17, wherein administration of the effective amount of the vasodilator results in increased force vital capacity in the subject.

19. The method of any one of embodiments 1-18, wherein the subject has mean pulmonary arterial pressure (mPAP)≥25 mmHg and a pulmonary arterial wedge pressure (PAWP)≤15 mmHg as confirmed by a baseline right heart catheterization (RHC).

20. The method of any one of embodiments 1-19, wherein the subject is suffering from interstitial pneumonia.

Other embodiments are set forth in the following claims.

### REFERENCES

1) Humbert M, Kovacs G, Hoeper M M, Badagliacca R, Berger R M F, Brida M, et al. 2022 ESC/ERS Guidelines for the diagnosis and treatment of pulmonary hypertension. Eur Heart J 2022; 43:3618-731.
2) Lee C, Hamlyn J, Porcelli J, Schmit A. Practical considerations in the management of inhaled prostacyclin therapy for pulmonary hypertension associated with interstitial lung disease (WHO group 3). Respir Med 2022; 196:106806.
3) Tanabe N, Kumamaru H, Tamura Y, Taniguchi H, Emoto N, Yamada Y. et al. Multi-Institutional Prospective Cohort Study of Patients With Pulmonary Hypertension Associated With Respiratory Diseases. Circ J 2021; 85:333-42.
4) Nathan S D, Behr J, Collard H R, Cottin V, Hoeper M M, Martinez F J, et al. Riociguat for idiopathic interstitial pneumonia-associated pulmonary hypertension (RISE-IIP): a randomised, placebo-controlled phase 2b study. Lancet Respir Med 2019; 7:780-90.
5) Raghu G, Behr J, Brown K K, Egan J J, Kawut S M, Flaherty K R, et al. Treatment of idiopathic pulmonary fibrosis with ambrisentan: a parallel, randomized trial. Ann Intern Med 2013; 158:641-9.
6) Mitchell J A, Ahmetaj-Shala B, Kirkby N S, Wright W R, Mackenzie L S, Reed D M, et al. Role of prostacyclin in pulmonary hypertension. Glob Cardiol Sci Pract 2014; 2014:382-93.
7) Kuwana M, Abe K, Kinoshita H, Matsubara H, Minatsuki S, Murohara T, et al. Efficacy, safety, and pharmacokinetics of inhaled treprostinil in Japanese patients with pulmonary arterial hypertension. Pulm Circ 2023; 13:e12198.
8) Waxman A, Restrepo-Jaramillo R, Thenappan T, Ravichandran A, Engel P, Bajwa A, et al. Inhaled Treprostinil in Pulmonary Hypertension Due to Interstitial Lung Disease. N Engl J Med 2021; 384:325-34.
9) Corte T J, Keir G J, Dimopoulos K, Howard L, Corris P A, Parfitt L, et al. Bosentan in pulmonary hypertension associated with fibrotic idiopathic interstitial pneumonia. Am J Respir Crit Care Med 2014; 190:208-17.
10) Corte T J, Wort S J, Gatzoulis M A, Macdonald P, Hansell D M, Wells A U. Pulmonary vascular resistance predicts early mortality in patients with diffuse fibrotic lung disease and suspected pulmonary hypertension. Thorax 2009; 64:883-8.
11) Chebib N, Mornex J F, Traclet J, Philit F, Khouatra C, Zeghmar S, et al. Pulmonary hypertension in chronic lung diseases: comparison to other pulmonary hypertension groups. Pulm Circ 2018; 8:2045894018775056.
12) Sato T, Furukawa T, Teramachi R, Fukihara J, Yamano Y, Yokoyama T, et al. Mild elevation of pulmonary vascular resistance predicts mortality regardless of mean pulmonary artery pressure in mild interstitial lung disease. Thorax 2024; 79:422-9.
13) Wakamatsu K, Nagata N, Kumazoe H, Oda K, Ishimoto H, Yoshimi M, et al. Prognostic value of serial serum KL-6 measurements in patients with idiopathic pulmonary fibrosis. Respir Investig 2017; 55:16-23.
14) Nathan S D, Waxman A, Rajagopal S, Case A, Johri S, DuBrock H, et al. Inhaled treprostinil and forced vital capacity in patients with interstitial lung disease and associated pulmonary hypertension: a post-hoc analysis of the INCREASE study. Lancet Respir Med 2021; 9:1266-74.

1. A method of treating a subject having increased expression of KL-6 and suffering from lung disease or lung dysfunction comprising administering an effective amount of a vasodilator to the subject.

2. A method of treating lung disease or lung dysfunction in a subject suffering therefrom and having an increased expression of KL-6, comprising administering an effective amount of a vasodilator to the subject.

3. A method of reducing KL-6 expression in a subject suffering from lung disease or lung dysfunction, comprising administering an effective amount of a vasodilator to the subject.

4. A method of treating lung disease or lung dysfunction in a subject suffering therefrom and having previously been determined as having increased expression of KL-6, comprising administering an effective amount of a vasodilator to the subject.

5. The method of claim 1, wherein the vasodilator comprises Treprostinil or a salt thereof.

6. The method of claim 1, wherein the effective amount of the vasodilator is an amount sufficient to reduce KL-6 expression.

7. The method of claim 1, wherein the lung disease comprises pulmonary hypertension associated with interstitial lung disease (PH-ILD).

8. The method of claim 1, wherein the patient exhibits a baseline expression of KL-6 that is greater than 600 U/ml, or greater than 650 U/ml, or greater than 700 U/ml, or greater than 750 U/ml, or greater than 800 U/ml prior to administration of the vasodilator.

9. The method of claim 8, wherein the effective amount of vasodilator is an amount sufficient to decrease KL-6 expression to below 600 U/ml, or below 650 U/ml, or below 700 U/ml or below 750 U/ml.

10. The method of claim 1, wherein the vasodilator is administered by intravenous administration, intramuscular administration, oral administration, or inhalation.

11. The method of claim 1, wherein the subject is a mammalian subject, optionally, wherein the subject is a human subject.

12. The method of claim 1, wherein the Treprostinil or salt thereof is administered by inhalation at a concentration of about 500 μg/ml to about 2500 μg/ml.

13. The method of claim 1, wherein the subject exhibits a baseline forced vital capacity of less than 2.20 L, or less than 2.15 L, or less than 2.10 L prior to administration of the vasodilator.

14. The method of claim 6, wherein the effective amount of the vasodilator is an amount sufficient to reduce KL-6 expression and increase forced vital capacity above 2.05 L, or 2.1 L, or 2.15 L, or 2.2 L.

15. The method of claim 5, wherein the effective amount is between 5-500 μg inhaled treprostinil per day.

16. The method of claim 1, further comprising the step of detecting the level of KL-6 expression prior to administration of the vasodilator.

17. The method of claim 1, further comprising the step of detecting the level of KL-6 expression following administration of the vasodilator.

18. The method of claim 1, wherein administration of the effective amount of the vasodilator results in increased force vital capacity in the subject.

19. The method of claim 1, wherein the subject has mean pulmonary arterial pressure (mPAP)≥25 mmHg and a pulmonary arterial wedge pressure (PAWP)≤15 mmHg as confirmed by a baseline right heart catheterization (RHC).

20. The method of claim 1, wherein the subject is suffering from interstitial pneumonia.
